(19) United States
(12) Patent Application Publication
(10) Pub. No.: US 2008/0065014 A1
(43) Pub. Date: Mar. 13, 2008

(54) Systems, Methods, and Devices to Facilitate Wire and Device Crossings of Obstructions in Body Lumens

(75) Inventors: Randolf Von Oepen, Los Altos Hills, CA (US); Kelly J. McCrystle, Menlo Park, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Abbott Park, IL (US)

(21) Appl. No.: 11/738,378

(22) Filed: Apr. 20, 2007

Related U.S. Application Data
(60) Provisional application No. 60/793,781, filed on Apr. 21, 2006.

(51) Int. Cl. A61M 29/00

(52) U.S. Cl. 604/104

(57) ABSTRACT

In one of many possible embodiments, a method for treating an obstruction within a blood vessel includes applying a stimulus to a fluid near the obstruction to disrupt the obstruction. The stimulus may include applying a stimulus to the fluid near the obstruction to breach a proximal cap and applying a stimulus to dilate micro-channels formed within the obstruction. Such stimuli may include causing cavitation within one or more fluid near the device, expanding one or more fluid that is in contact with irregularities in the obstruction, and bombarding the obstruction with particles that undergo a rapid phase change.

### CROSS-REFERENCE TO RELATED APPLICATIONS

This application claims priority to and the benefit of U.S. Provisional Patent Application Ser. No. 60/793,781, filed Apr. 21, 2006, and entitled “Medical Devices,” the disclosure of which is incorporated herein by this reference.

### BACKGROUND OF THE INVENTION

I. Field of the Invention

The present invention generally relates to the field of medical devices. More specifically, the present invention relates to systems, methods, and devices for treating obstructions in a body lumen.

II. Related Technology

Cardiovascular disease is a leading cause of death worldwide. Consequently, many efforts have been directed at treating cardiovascular disease. One of the remaining frontiers of interventional cardiology is the treatment of chronic total occlusions (CTOs). CTOs are nearly complete blockages of arteries that often contain a fibrous or calcified proximal cap and micro-channels that span the occlusion length. Some approaches for treating a CTO make use of a guidewire that is moved into contact with the CTO. The guidewire is then forced through the CTO. There are, however a number of difficulties with this procedure.

One difficulty in treating these types of diseases partially lies within the trouble in finding a passage through the occlusion using a guidewire, and the potential vessel dissections that can occur when a guidewire is tracked away from an appropriate passage toward the vessel wall. For instance, it can be difficult to pass the guidewire through the proximal cap, which can result in the guidewire being directed off-track and through the vessel.

Further, with the proximal cap being often formed of fibrous or calcified material, it is generally difficult to breach the cap and access the distal side of the CTO with a guidewire. Accordingly, if pushing the guidewire distally fails to breach the proximal cap and/or the main portion of a CTO, the distal side access is prevented and other medical procedures are necessary. This results in increased costs and time to perform the desired procedure.

It would be advantageous to have a device that can facilitate passage of the guidewire through a CTO or other obstruction within a body lumen. In this manner, the devices, with associated systems and methods, can increase the effectiveness of accessing the CTO and its distal side for performance of a procedure.

### BRIEF SUMMARY OF THE INVENTION

In one of many possible embodiments, a method for treating an obstruction within a blood vessel includes applying a stimulus to a fluid near the obstruction in order to disrupt the obstruction. The method may include applying a stimulus to the fluid near the obstruction to breach a proximal cap and applying a stimulus to dilate micro-channels formed within the obstruction. Such stimuli may include causing cavitation within a fluid near the device, expanding a fluid that is in contact with irregularities in the obstruction, and bombarding the obstruction with particles that undergo a rapid phase change.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings.

FIG. 1 is a flowchart illustrating a method of crossing an obstruction in a vessel according to one example;

FIG. 2A is a cross-sectional view of a vessel in which an expandable member has been expanded to seal the vessel relative to the obstruction, and in which a fluid is introduced according to one example;

FIG. 2B is a cross-sectional view of a vessel in which a catheter is in proximity to the proximal cap of the obstruction according to one example;

FIG. 2C is a schematic diagram of a device for treating an obstruction in a vessel, such as a chronic total occlusion according to one example;

FIG. 2D is a cross-sectional view of a vessel in which energy has been applied to cause cavitation within the vessel to breach the proximal cap of an obstruction according to one example;

FIG. 2E is a cross-sectional view of a vessel with the expansion of micro-channels according to one example;

FIG. 2F is a cross-sectional view of a vessel with a guidewire crossing the obstruction according to one example;

FIG. 3 is a cross-sectional view of a vessel in which bursting particles are bombarding an obstruction to breach the proximal cap according to one example;

FIG. 4 is a cross-sectional view of a vessel having an obstruction in which air-bearing particles are introduced into the vessel according to one example;

FIG. 5A is a cross-sectional view of a vessel in which an expandable fluid is introduced to an obstruction and enters micro-channels within the obstruction according to one example;

FIG. 5B is a cross-sectional view of a vessel in which the expandable fluid is expanded within the micro-channels to thereby increase the size of the micro-channels according to one example; and

FIG. 5C is a cross-sectional view of a vessel with a guidewire crossing the obstruction according to one example.

The accompanying drawings illustrate various embodiments of the present system and method and are a part of the specification. The illustrated embodiments are merely examples of the present system and method and do not limit the scope of the disclosure. Throughout the drawings, identical reference numbers designate similar, but not necessarily identical, elements.

### DETAILED DESCRIPTION

Systems, methods, and devices are provided herein for crossing obstructions formed within the vasculature or body lumen of a patient, such as chronic total occlusions. Such obstructions frequently include a proximal cap that is fibrous and/or calcified that at least partially covers the obstruction's remaining main portion. The main portion can include micro-channels or micro-cracks formed therein. The systems, methods, and devices described herein are configured to breach the proximal cap and to expand the micro-channels or micro-cracks to allow a guidewire or other device to pass through the expanded micro-channels or micro-cracks to cross the obstruction.

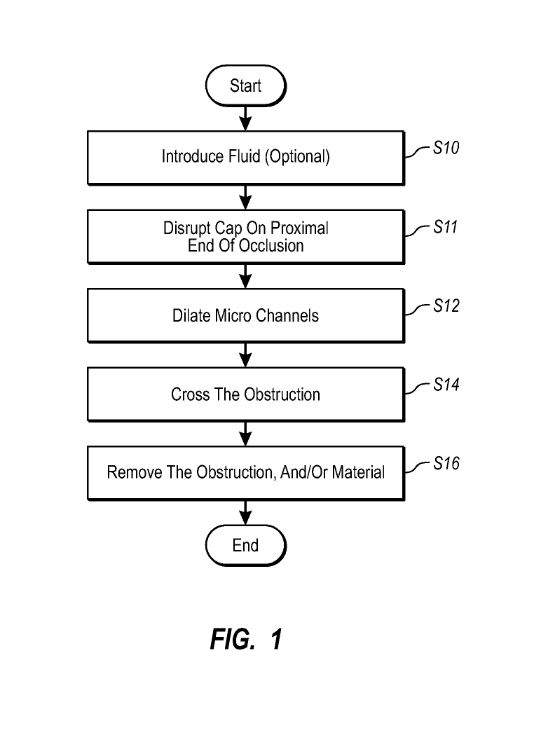

FIG. 1 is a flowchart illustrating a generalized method of crossing an obstruction in a vessel according to one example. The method may optionally begin by introducing a fluid into the body lumen, such as a vessel in close proximity to an obstruction, as represented by block S10. In one example, cavitation if caused by subjecting the fluid to a stimulus, the cavitation causing erosion or degradation of the cap or micro-channels. In another example, the fluid may be a fluid that comes into contact with the obstruction in a relatively unexpanded state and expands in response to a stimulus. Various manners are known to deliver a fluid within a body lumen. Those methods, systems, and devices used to deliver a contrast medium may be used. For instance, the fluid can be injected through a guiding catheter using a syringe, endoinflator or powered pump. Additionally, a microcatheter, a needle, a micro-needle or similar devices may be utilized to deliver a fluid to the desired site. Other devices, methods, and systems for injecting the fluid are known to those skilled in the art.

With continued reference to FIG. 1, the method continues by disrupting a proximal cap of the obstruction, as represented by block S11. In particular, the disruption may be sufficient to breach the proximal cap. The disruption may be caused by any number of factors and/or combination of factors. Some factors may include, without limitation cavitation, rapid phase change of a solid to a gas, solid to liquid, and/or liquid to a gas, or, expansion of an expandable fluid, and/or any combination of the above. Further details of one or more systems, methods, and devices to disrupt the cap are described herein.

Once the proximal cap has been disrupted, the method continues by dilating the micro-channels are dilated within the obstruction, as represented by block S12. One or more of the factors described above may be used to expand the micro-channels within the obstruction. Following micro-channel dilation, a guidewire or other medical device may then be moved through the micro-channels to thereby cross the obstruction, as represented by block S14. With the guidewire or other medical device having been passed through the obstruction, the obstruction can be dilated or at least partially removed from the vessel lumen, as represented by block S16. The present method describes a generalized process for crossing an obstruction within a vessel according to one example. Several examples will be discussed in more detail below, beginning with a discussion of one exemplary method of using methods, systems, or devices, to create cavitation.

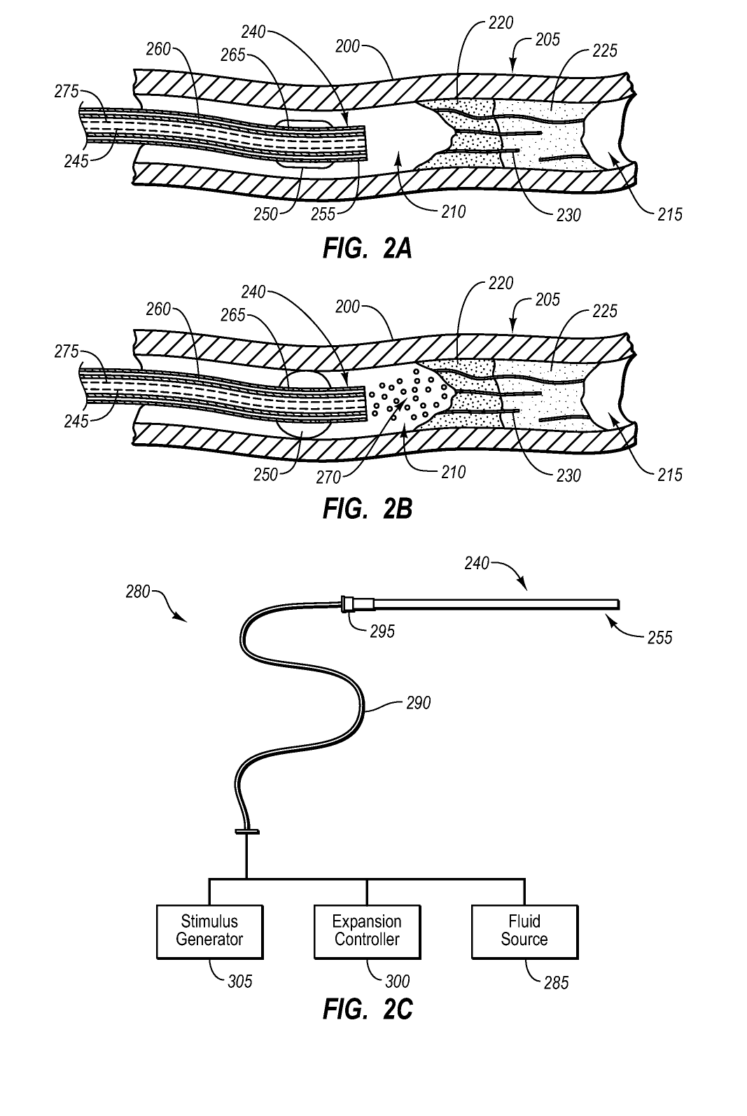

FIG. 2A is a cross-sectional view of a vessel 200 within which the above-described method may be performed. An obstruction 205 is illustrated as being located within the vessel 200. The obstruction 205 may partially or fully restrict the flow of blood through the vessel 200. In the illustrated example, the obstruction 205 may be a chronic total occlusion, which prevents a substantial portion or even all of the blood in the vessel from passing from a proximal side 210 of the vessel 200 through to a distal side 215 of the vessel 200.

The obstruction 205 may include a proximal cap 220 on the proximal side 210 of the vessel 200. The proximal cap 220 may be relatively hard and/or fibrous relative to a main portion 225 of the obstruction 205. The nature of the proximal cap 220 may make it relatively difficult for a medical device, such as a guidewire, to pass through the proximal cap 220 to thereby treat the obstruction 205. The obstruction 205 as well as the proximal cap 220 may also include micro-channels or micro-cracks 230 formed therein. Further, the main portion 225 of the obstruction 205 may be generally softer than the proximal cap 220.

As illustrated in FIG. 2A, during the procedure a catheter 240 is brought into proximity to the proximal cap 220 of the obstruction 205. The catheter 240 may be brought into proximity with the obstruction 205 by any suitable method, including the use of a guide catheter and a guide wire (not shown) disposed within a lumen 245 of the catheter 240. The catheter 240 will be described in the context of accessing the obstruction 205 from the proximal side 210 of the vessel 200. The catheter 240 may alternatively be used to access the obstruction 205 from the distal side 215 of the vessel 200 as desired.

Continuing with the illustrated example, the catheter 240 optionally includes an expandable member 250, such as an inflatable balloon, disposed near or at a distal end 255 of the catheter 240. The expandable member 250 is in fluid communication with a fluid lumen 260 formed in the catheter 240 through ports 265. An expansion fluid can be delivered along the fluid lumen 260 from a proximal end of the catheter 240 using a syringe, pump, or other device typically used to deliver fluid to an expandable or inflatable balloon. The catheter 240, therefore, can function and operate similarly to a balloon catheter. The expandable member 250 may be selectively expanded to provide a seal between the vessel 200 and the expandable member 250. FIG. 2B illustrates the expandable member 250 expanded to seal the vessel 200 relative to the obstruction 205 according to one example.

According to the present example, and with reference to FIG. 2B, a fluid 270 may be introduced into the vessel 200 near the obstruction 205 after the vessel 200 has been sealed by expansion of the expandable member 250. For instance, the fluid 270 can be introduced along lumen 245 of the catheter 240. Alternatively, a fluid delivery lumen 275 can be formed within the catheter 240, as represented by the dotted line, and used to deliver the fluid 270 from the distal end of the catheter 240.

Sealing the vessel 200 prior to introduction of the fluid 270 may drive the fluid 270 into irregularities within the obstruction 205, such as surface irregularities and/or micro-channels in the proximal cap 220 and/or the main portion 225. One suitable fluid may include a gas, such a carbon dioxide. Accordingly, the fluid 270 illustrated is shown as bubbles. Although illustrated as bubbles, in one or more examples, if a gas is the fluid 270 introduced, the gas 270 may be dissolved in the fluid disposed in the lumen, such as blood. Therefore, it may be possible to use the gas 270 dissolved within the blood to expand the mirco-channels 230.

Though gas is referenced, any fluid may optionally be introduced, whether the fluid is a liquid state, a gaseous state, semi-solid, or any combination thereof Other suitable fluids may include contrast media, saline or other biocompatible fluids.

FIG. 2C schematically illustrates a system 280, which includes and utilizes the catheter 240. As illustrated, and suggested above, the catheter 240 is in fluid communication with at least one fluid source 285 to provide one or more fluids as described above. This fluid communication can be achieved through various medical grade tubular members 290, such as catheters, etc, with associated fluid sealing connections 295, such as luer lock connections or the like. For instance, the fluid source 285 can provide the fluid delivered through the distal end 255 of the catheter 240 to drive, penetrate or infiltrate the proximal cap 220 of the obstruction. In addition to, or alternatively, the fluid source 285 can provide the fluid used to expand or inflate the expandable member 240. The fluid source 285 can be, therefore, the reservoir or tanks holding the fluid, whether alone or in combination with pumps or other devices usable to deliver the fluid to the catheter 240 and eventually the obstruction 205 (FIG. 2A)

The system 280 may further include or be coupled to an expansion controller 300, which may allow a user to selectively expand the expandable member 250 (FIG. 2A) to selectively seal the vessel 200 (FIG. 2A), as described above. The expansion controller 300 can use the fluid from the fluid source 285 to expand the expandable member 250 or can include a separate fluid reservoir or tank for delivering fluid to the expandable member 250. The expansion controller 300 can also fluidly communicate with the catheter 240 using medical grade tubular members 290 with associated fluid sealing connections 295.

As illustrated in FIG. 2C, the catheter 240 may be coupled to a stimulus generator 305, such as a cavitation stimulus generator. The stimulus generator 305 may be configured to provide any number of stimuli as desired. If the stimulus generator 305 is a cavitation stimulus generator, the stimulus generator 305 may be configured to generate a stimulus to cause cavitation near the distal end 255 of the catheter 240. Cavitation stimuli may include, without limitation, ultrasonic energy, vibration, sound waves, light, heat, other electromagnetic energy or any other form of stimuli for causing cavitation at or near the distal end 255 of the catheter 240. The fluid 270 introduced from expansion controller 300 and/or fluid source 285 may be used to enhance the effectiveness of cavitation within the vessel in disrupting the obstruction 205.

FIG. 2D is a cross-sectional view of the vessel 200 in which energy is applied to cause cavitation within the vessel 200 to breach the proximal cap 220 of the obstruction 205. In particular, a cavitation stimulus is applied to the distal end 255 of the catheter 240. The cavitation stimulus in one example may be ultrasonic energy. In the illustrated example, a stimulus delivery mechanism 310, such as an ultrasonic energy transducer disposed at or near the distal end 255 of the catheter 240, may be used to apply ultrasonic energy to the treatment site. The stimulus delivery mechanism 310 may be tracked through the catheter 240, such as through the lumen 245 or one of the other lumens as desired. In addition, the stimulus delivery mechanism 310 may be coupled to the stimulus generator 305 (FIG. 2C).

Within most liquids, including blood, there may be continuous transition of the movement of the liquid as a sound wave passes therethrough, as long as the amplitude or “loudness” of the sound is relatively low. As amplitude is increased, however, such as to the level of ultrasonic energy, the magnitude of the negative pressure in the areas of rarefaction eventually becomes sufficient to cause the liquid to fracture because of the negative pressure. Cavitation voids or “bubbles” are created at sites of rarefaction as the liquid fractures or tears because of the negative pressure of the sound wave in the liquid.

As the wave fronts pass, the cavitation bubbles oscillate under the influence of positive pressure, eventually growing to an unstable size. Finally, the violent collapse of the cavitation bubbles results in implosions, which cause shock waves to be radiated from the sites of the collapse. The collapse and implosion cavitation bubbles throughout an ultrasonically activated liquid result in the effect commonly associated with ultrasonic energy.

The cavitation can be directed at the blood contacting the proximal cap 220 of the obstruction 205. Therefore, as the bubbles implode, forces will be applied to the proximal cap 220 that will result in cracking of any fibrous or calcified material. As previously introduced, the fluid 270, may be driven into the irregularities within the obstruction 205. The application of a cavitation stimulus near the obstruction causes the fluid in the irregularities to cavitate. This cavitation may thus enhance the disruption of the obstruction 205 due to cavitation, including the proximal cap 220. Further, the disruption may expand the micro-channels 230 in the main portion 225 of the obstruction. In addition, the steps of injecting fluid and inducing cavitation can be alternated step-wise in order to advance the micro-channel expansion across the obstruction 205 and enable guidewire access and crossing.

One example of the disruption to the obstruction is illustrated in FIG. 2E in which the proximal cap 220 has been breached and the micro-channels 230 in the main portion 225 have been dilated. After the proximal cap 220 has been breached and the micro-channels 230 have been dilated, the expandable member 250 may be collapsed and the catheter 240 withdrawn.

Next, as illustrated in FIG. 2F, a guidewire 315 or other instrument may be used to cross the obstruction 205 through the dilated micro-channels 230. In one example a catheter is used in cooperation with a stimulus delivery mechanism 310 that is capable of directing ultrasonic energy into the blood stream, and thereby creating cavitation within the blood. These resulting cracks provide pathways by which a guidewire can be tracked into the obstruction in order to attempt a crossing of the obstruction.

Accordingly, FIGS. 2A-2F illustrate one example of a device that makes it possible to breach the proximal cap 220 of an obstruction 205 with micro-channels 230, and to dilate these micro-channels 230, thereby providing a pathway that may be accessed by a guidewire and subsequently tracked through. Further, this example is one more detailed example of the generalized process discussed with reference to FIG. 1 that includes the broad steps of optionally introducing a fluid, disrupting a proximal cap, and dilating micro-channels.

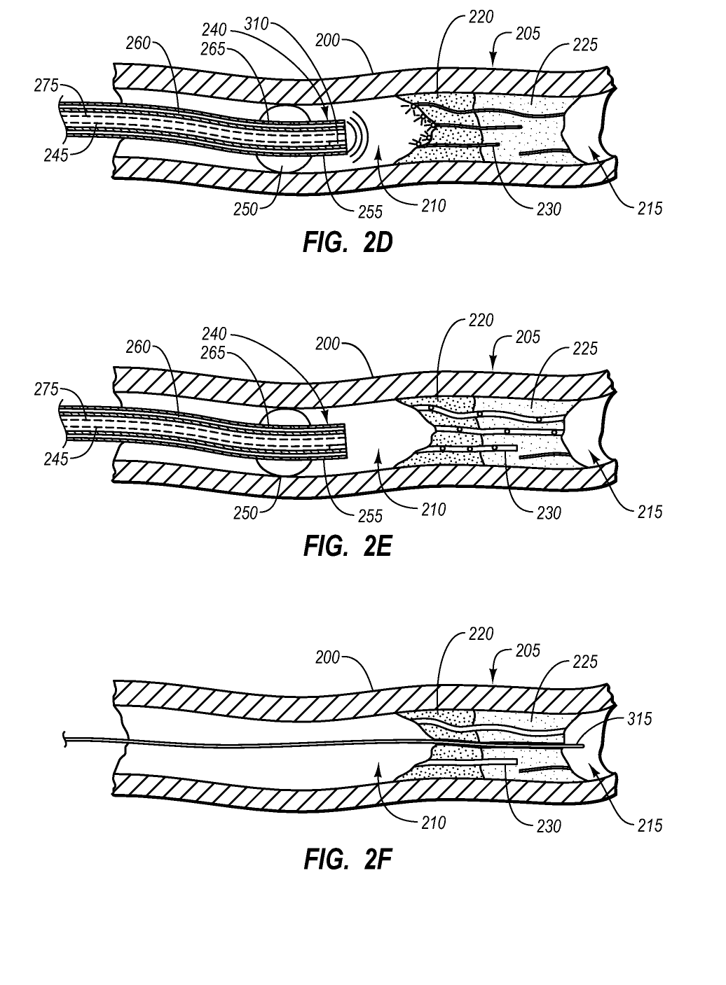

Other more specific examples may also be provided for accomplishing one or more of the steps described above. FIG. 3 is a cross-sectional view of a vessel 200 in which particles 350, which undergo a rapid phase change, are bombarding an obstruction 205 to breach the proximal cap 220 according to one example. The particles 350 may be released from a distal end 255′ of a catheter 240′ near the proximal cap 220. In particular, the particles 300 may be released through any of the lumens 245, 260, and/or 275. The particles 350 directed to the obstruction 205 may go through a rapid phase change in response to a stimulus or through entering the blood stream. In one example, such a stimulus may include the particles 350 contacting the proximal cap 220 or the blood.

The particles 350 may be introduced through the distal end 255 of the catheter and may be provided by an outside source. Accordingly, in the present case the stimulus generator 305 illustrated in FIG. 2C may be configured to deliver the particles 350. The particles 350 may also be responsive to other stimuli, such as vibrational, thermal, or any other stimulus or combination of stimuli. Such stimuli may be provided by a stimulus delivery mechanism 310, such as illustrated in FIG. 2D. As the particles 350 undergo a rapid phase change, they disrupt the surrounding area. Accordingly, by bombarding the obstruction 205, the particles 350 may disrupt the obstruction 205 as they undergo a rapid phase change to breach the proximal cap 220 and expand the micro-channels 230. Further, those particles 350 that enter the micro-channel 230 can undergo the rapid phase change and increase the dimensions of the micro-channels 230. One type of material that may be used for the particles 350 can include, but are not limited to, crystals of solid carbon dioxide that can be delivered to the CTO.

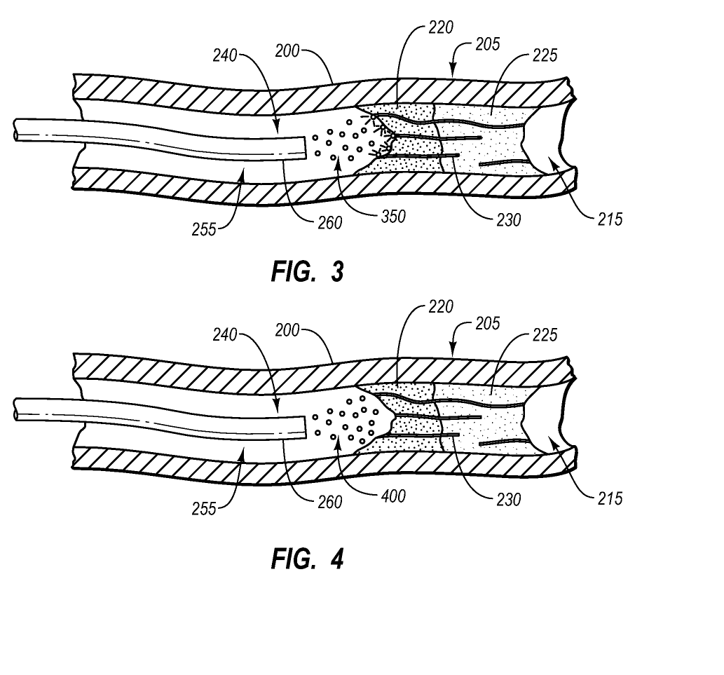

FIG. 4 is a cross-sectional view of a vessel having an obstruction 205 in which a fluid, such as an expandable gas 400, is allowed to expand within the vessel 200. As illustrated in FIG. 4, the expandable gas 400 may be introduced through the catheter 240 to the obstruction 205. In particular, the expandable gas 400 may be introduced through the fluid delivery lumen 275. Upon entry in the blood stream within the vessel 200 and within the micro-channels 230 in particular, the expandable gas 400 begins to expand in order to reach equilibrium with the surrounding pressure as the expandable gas 400 is being absorbed by the blood in the vessel 200.

The expansion of the expandable gas 400 will pressurize and expand the micro-channels. After the expandable gas 400 is absorbed into the blood stream, the micro-channels 230 would maintain an increased diameter, which would aid in accessibility of crossing the obstruction 205 with a guidewire 315 or other instrument. The expandable gas 400 may include any type of gas, including carbon dioxide. Further, while illustrated separately from a cavitation process, the use of expanding gas may be used in concert with a cavitation process. Other processes may also be used to breach the proximal cap 220 and/or dilate the micro-channels 230.

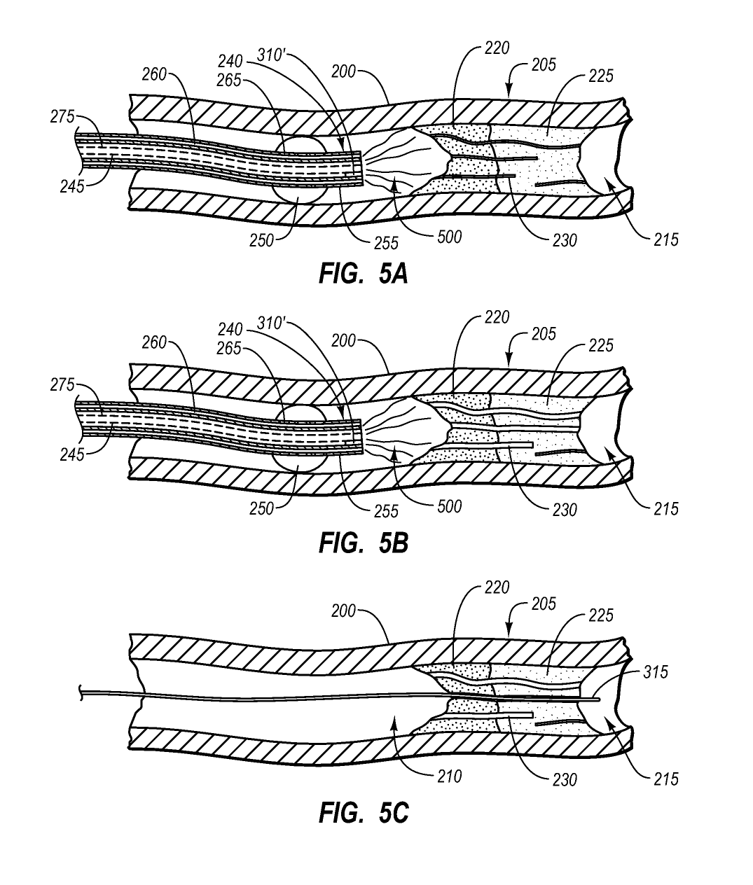

FIG. 5A is a cross-sectional view of a vessel 200 in which an expandable fluid 500 is introduced into an obstruction 205. In particular, the expandable fluid 500 may be delivered through a distal end 255 of a catheter 240, which may be part of a system 280 (not shown) similar to that illustrated in FIG. 2C. In particular, the expandable fluid 500 may be introduced through the fluid delivery lumen 275. The catheter 240 may include an expandable member 250 coupled thereto that may be selectively expanded to seal the vessel 200. The expandable member 250, shown expanded in FIG. 5A, may be expanded before the expandable fluid 500 is delivered through the distal end 255 of the catheter 240. The expandable member 250 is in fluid communication with a fluid lumen 260 formed in the catheter 240 through ports 265. An expansion fluid can be delivered along the fluid lumen 260 from a proximal end of the catheter 240 using a syringe, pump, or other device typically used to deliver fluid to an expandable or inflatable balloon. The catheter 240, therefore, can function and operate similarly to a balloon catheter.

Once the expandable fluid 500 has been delivered to the obstruction 205, the expandable fluid 500 enters micro-channels 230 or other irregularities within the obstruction 205 according to one example. If the expandable member 250 is expanded to seal the vessel 200, the expandable fluid 500 may be delivered at a relatively higher pressure. Once the expandable fluid 500 has penetrated the micro-channels 230, the expandable fluid 500 may be expanded in response to a stimulus to thereby dilate the micro-channels 230.

FIG. 5B is a cross-sectional view of the vessel 200 in which the expandable fluid 500 is expanded within the micro-channels 230 to thereby dilate the micro-channels according to one example. The expandable fluid 500 may expand in response to various stimuli. For example, the expandable fluid 500 can expand as a result of thermal stimuli. The thermal stimuli may be applied by a stimulus delivery mechanism 310′. The thermal stimulus delivery mechanism 310′ may be tracked through the catheter 240, such as through the lumen 245 or one of the other lumens as desired.

In particular, water may be the expandable fluid 500. If the fluid used is saline, then a cryogenic agent can be introduced using the stimulus delivery mechanism 310′ in the catheter to the treatment site in order to freeze the fluid. Since saline expands when it is frozen, the fluid expansion would result in an expansion of the micro-channels 230. The thermal energy in the vessel 200 and the surrounding areas would then cause the saline to melt and become dispersed in the blood stream. However, the then dilated micro-channels 230 would remain dilated. This process can then be repeated several times in order to force the enlargement of the micro-channels 230 across the entire length of the obstruction 205.

Another thermal stimulus may include heating a fluid that expands in response to heat. Therefore, another process could include heating of the expandable fluid 500 with the stimulus delivery mechanism 310′ at a level that is safe to the patient. Expanding the expandable fluid 500 would therefore dilate the micro-channels 230 as described above. Once the micro-channels 230 and the proximal cap 220 have been dilated, a guidewire 315 or other instrument may then be introduced to cross the obstruction 205, as illustrated in FIG. 5C. It is further contemplated that the expandable fluid may be a fluid that undergoes a phase change, for example the material could be delivered in a fluid state, whereby through a chemical reaction such as through mixing during the delivery process, the material may undergo a chemical reaction causing a volume change. Exemplary materials can be expandable foams either open or closed cell.

The preceding description has been presented only to illustrate and describe exemplary embodiments. It is not intended to be exhaustive or to limit the disclosure to any precise form disclosed. Many modifications and variations are possible in light of the above teaching. It is intended that the scope of the disclosure be defined by the following claims.

What is claimed is:

1. A method for treating an obstruction within a blood vessel, the method comprising applying a stimulus to a fluid near the obstruction to disrupt the obstruction.

2. The method of claim 1, wherein applying a stimulus to a fluid near the obstruction to disrupt the obstruction includes applying a stimulus to the fluid near the obstruction to breach a proximal cap and applying a stimulus to dilate micro-channels formed within the obstruction.

3. The method of claim 2, wherein applying a stimulus to the fluid to breach the proximal cap and applying a stimulus to the fluid to dilate micro-channels formed within the obstruction include applying a stimulus to separate stimuli.

4. The method of claim 1, wherein applying a stimulus to a fluid near the obstruction to disrupt the obstruction includes applying a cavitation stimulus.

5. The method of claim 4, wherein applying the cavitation stimulus includes applying at least one of ultrasonic energy, thermal energy, vibrational energy, and light energy.

6. The method of claim 5, further comprising delivering a fluid near the obstruction.

7. The method of claim 6, wherein delivering the fluid near the obstruction includes delivering a gas.

8. The method of claim 6, further comprising sealing the blood vessel to form a sealed portion of the vessel.

9. The method of claim 8, wherein delivering the fluid near the obstruction includes delivering the fluid to the sealed portion within the vessel.

10. The method of claim 9, wherein delivering the obstruction to the sealed portion includes increasing a pressure in the sealed portion of the vessel.

11. The method of claim 1, wherein applying a stimulus to disrupt the obstruction includes delivering an expandable fluid into irregularities formed within the obstruction and applying an expansion stimulus to expand the expandable fluid.

12. The method of claim 11, wherein delivering the expandable fluid includes delivering an expandable liquid.

13. The method of claim 12, wherein delivering the expandable liquid includes delivering water and applying an expansion stimulus.

14. The method of claim 11, wherein delivering the expandable fluid includes delivering an expanding gas.

15. The method of claim 14, further comprising sealing the blood vessel to form a sealed portion of the vessel and delivering the expanding gas to the sealed portion within the vessel increasing a pressure in the sealed portion of the vessel.

16. The method of claim 14, wherein applying an expansion stimulus to the expanding gas includes applying heat to the expanding gas.

17. The method of claim 1, wherein disrupting the obstruction includes bombarding the obstruction with particles which undergo a rapid phase change.

18. A method for treating a chronic total occlusion (CTO) within a blood vessel, the method, comprising:
advancing a catheter within the vessel such that a distal portion of the catheter is near the CTO;
using the catheter to apply a stimulus to at least one fluid near the CTO to thereby breach a distal cap of the CTO.

19. The method of claim 18, further comprising applying a stimulus to at least one fluid near the CTO to dilate micro-channels formed within the channels.

20. The method of claim 18, wherein applying the stimulus includes at least one of providing a cavitation stimulus, bombarding the CTO with particles that undergo a rapid phase change, and expanding expandable fluid when the fluid contact is contact with irregularities formed in the CTO.

21. The method of claim 18, further comprising applying a stimulus to at least one fluid near the CTO to dilate micro-channels formed within the channels, the stimulus including at least one of providing a cavitation stimulus, bombarding the CTO with particles that undergo a rapid phase change, and expanding expandable fluid when the fluid contact is contact with irregularities formed in the CTO.

22. A device for treating an obstruction within a blood vessel, the device comprising:
a catheter having a distal end and a proximal end,
at least one stimulus generator coupled to a proximal end of the catheter, wherein the stimulus generator is configured to apply at least one stimulus via the distal end of the catheter to at least one fluid near the obstruction to thereby disrupt the obstruction.

23. The device of claim 22, wherein the stimulus generator is configured to apply a cavitation stimulus to fluid near the obstruction.

24. The device of claim 23, wherein the stimulus includes an ultrasonic energy source.

25. The device of claim 23, wherein the stimulus generator is configured to apply a cavitation stimulus that includes at least one of an ultrasonic stimulus, a thermal stimulus, a light stimulus, and a vibrational stimulus.

26. The device of claim 22, further comprising a fluid source coupled to a proximal end of the catheter, the fluid source being configured to deliver a fluid through a distal end of the catheter.

27. The device of claim 26, wherein the fluid source is configured to deliver a gas through the distal end of the catheter.

28. The device of claim 26, further comprising an expandable member coupled to a distal end of the catheter and an expansion controller operably coupled to the expandable member, the expansion controller being configured to selectively expand the expandable member to seal a wall of the vessel.

29. The device of claim 28, wherein the fluid source is configured to deliver the fluid at a relatively high pressure to thereby increase the pressure between the expandable member and the instruction.

30. The device of claim 22, wherein the stimulus generator is configured to apply a thermal stimulus.

31. The device of claim 29, further comprising a fluid source coupled to the catheter, the fluid source configured to deliver an expandable fluid through a distal end of the catheter device and wherein the stimulus generator is configured to selectively apply a thermal stimulus to expand the expandable fluid.

32. The device of claim 31, wherein the fluid source is configured to deliver water and the stimulus generator is configured to selectively apply a cryogenic stimulus to expand the water.

33. The device of claim 31, wherein the fluid source is configured to deliver an expandable gas and the stimulus generator is configured to selectively apply a heat stimulus to expand the expandable gas.
